(19) United States
(12) Patent Application Publication
(10) Pub. No.: US 2019/0022259 A1
(43) Pub. Date: Jan. 24, 2019

(54) Use of 99mTc-tilmanocept and related molecular constructs for identifying and diagnosing malignant tumors and for monitoring therapeutic anti-tumor interventions

(71) Applicant: Navidea Biopharmaceuticals, Inc., Dublin, OH (US)

(72) Inventors: Frederick O. Cope, Dublin, OH (US); Bonnie Chandler Abbruzzese, Dublin, OH (US); David Allen Ralph, Columbus, OH (US); Allison Kissling, Dublin, OH (US)

(21) Appl. No.: 16/041,673

(22) Filed: Jul. 20, 2018

Related U.S. Application Data
(60) Provisional application No. 62/535,752, filed on Jul. 21, 2017.

(51) Int. Cl. A61K 51/06

(52) U.S. Cl. CPC A61K 51/065

(57) ABSTRACT

The present application relates to compositions and methods for imaging and/or detecting tumors. In certain embodiments, the imaging and/or detection is targeted to tumor associated macrophages. In some embodiments, a mannosylated dextran construct is used to target and/or detect tumor associated macrophages. In some embodiments, the tumor may be one or more visceral tumors. In some embodiments, the detected and/or targeted tumor may be one or more metastatic tumors.

Macrophages can attain a wide range of activated phenotypes or can alter their activated phenotypes in response to stimuli from their local environments. When activated macrophages with varying phenotypes were first recognized, these differing activated phenotypic states were classified as either proinflammatory (which can be referred to as “activated” or “M1”) or wound-healing (which can be referred to as “alternatively activated”, “immunosuppressing” or “M2”) to parallel the Th1/Th2 paradigm of activated T helper cells. The activated phenotypes of macrophages can be highly diverse and a simple dichotomous classification of macrophage activation states as either M1 and M2 is not believed to accurately represent the richness of the phenotypic plasticity of which macrophages are capable.

Macrophages express a gene encoding the macrophage mannose receptor (CD206). Some other myeloid-derived cells besides macrophages, including myeloid-derived suppressor cells (MDSC) (and some dendritic cells) but not lymphocytes, also express CD206. Importantly, CD206 expression level can be altered and elevated significantly when macrophages attain certain activated phenotypic states. Generally, but not always or exclusively, macrophages with M2-like activated phenotypic states express high levels of CD206 relative to the CD206 expression levels observed in macrophages with an M1-like activated state. Tumor activated macrophages (TAMs) are most frequently M2-like and highly express CD206. Thus, tumors contain large numbers of cells—comprised of mostly TAMs, MDSCs, and possibly dendritic cells—that express high levels of the macrophage mannose receptor (CD206).

The macrophage mannose receptor (CD206) is a trans-membrane C-type lectin protein with 8 carbohydrate binding domains (CBDs). All of the CBDs bind mannose, but some of CD206's CBD also bind other sugars. It has been noted that single mannose molecules (mannose monomers) and monomers of sugars other than mannose have low affinity for CD206. Ligands with many mannose moieties that can interact with many CBDs on CD206 can form very high affinity interactions with CD206, and compositions described herein can thus have high affinity interactions with CD206 and macrophages expressing CD206. In some embodiments, a composition as described herein can have a high affinity for CD206 on TAMs. High affinity binding of CD206 and its ligands can rely on multivalent interactions between sugars displayed on the ligands and multiple CBDs on CD206.

### FIGURES

FIG. 1 shows a side view of a Balb/c mouse with a 4T1.2 tumor (T) 24 hours post injection with Cy5.5-tilmanocept. Auto-fluorescence from the intestines labeled (I).

FIG. 2 shows a dorsal view of a Balb/c mouse with a 4T1.2 tumor (T) 24 hours post injection with Cy5.5-tilmanocept.

FIG. 3 shows a dorsal view of a Balb/c mouse with a 4T1.2 tumor (T) 24 hours post injection with Cy5.5-tilmanocept.

FIG. 4 shows a side view of a Balb/c mouse with a 4T1.2 tumor (T). This animal was not injected with Cy5.5-tilmanocept (Auto-Fluorescence Control). Auto-fluorescence from the intestines labeled (I).

FIG. 5 shows a false color image showing the intensity of Cy5.5 fluorescence from the dissected liver (L), kidney (K), spleen (S) and tumor (T) from the Cy5.5-tilmanocept injected mouse that was shown in FIG. 1.

FIG. 6 shows the results achieved after administration of fludeoxyglucose (FDG) PET/CT compared with the results achieved after administration of ^{99m}Tc tilmanocept for a subject with colorectal cancer and disseminated liver metastases.

FIG. 7 shows the results achieved after administration of FDG PET/CT compared with the results achieved after administration of ^{99m}Tc tilmanocept for a second subject with colorectal cancer and disseminated liver metastases.

FIG. 8 shows an embodiment of a composition that can be used for the methods as described herein.

Tilmanocept® (Lymphoseek®, (99m)Tc-diethylenetriaminepentaacetic acid (DTPA)-mannosyl-dextran) is a mannosylated dextran molecular construct built on a 10 kD dextran backbone and conjugated with the chelating agent DTPA. Tilmanocept was specifically designed to be a high affinity ligand for CD206. Compositions embodied herein can have an average of approximately 17 mannoses and 5 DTPA moieties that are attached to a dextran backbone via amine terminated leashes, or other attachment mechanisms as described herein. Certain embodiments may comprise altering the spacing of any mannoses on a dextran backbone. For example, a mannose may be attached to every third dextran molecule. Embodiments described herein may comprise between 4 and 25 mannoses on a dextran backbone. Some embodiments may comprise more than 25 mannoses. In certain embodiments, valency and geometry of a glycoconjugate, such as those described herein, including but not limited to tilmanocept, may be altered to improve binding affinity to CD206. DTPA moieties can also permit tilmanocept to be efficiently labeled with various radioactive ions, including those of the gamma emitting metastable isotope of technetium, ^{99m}technetium, which has a half-life of 6.02 hours. Embodiments having multiple mannose moieties can enable high affinity, multivalent interactions with CD206 such as the high affinity interaction that have been observed between tilmanocept and CD206 (K_{D}=3×10⁻¹¹). After binding to CD206, compositions as described herein can be internalized into CD206 expressing cells by receptor mediated endocytosis.

Macrophages (and MDSCs) that highly express CD206 aggregate in, and are found to be abundant in, a variety of tissues involved in various disease processes. One such illness in which CD206 expressing cells are abundant is cancer, along with tumor-associated macrophages. Certain embodiments described herein use compositions for targeting CD206 expressing cells, such as cancer cells. As will be further described, by targeting these CD206 expressing cells, those cells can be imaged, quantified, and/or be treated using compositions described herein. Certain embodiments can comprise injecting a composition into the blood circulation (or released into the blood circulation by other means as described herein or as would be understood by one of skill in the art) that can enter tumors and localize to and internalize into CD206 expressing cells through a CD206-targeting composition. If the tumor localized tilmanocept is labeled with ^{99m}technetium, the tumor can be visualizable by various radiographic means due to the CD206-targeting composition localization of ^{99m}technetium in the tumor. Other radioactive isotopes, fluorescent markers, or other reporter groups could be substituted for ^{99m}technetium and provide similar radiographic imaging capabilities, which are described further herein. A radioisotope can be attached to a mannosylated dextran molecular construct using liker processes or chelation with DTPA, which would be understood by the skilled artisan.

Certain embodiments for preparing a composition described herein comprise attaching a radioactive isotope with a chelating agent (which can be DTPA or other chelating agents besides DTPA). Certain compositions may be prepared by attaching a radioactive isotope with a mannosylated dextran construct using an attaching process that is not chelation. Compositions described herein can be a mannosylated dextran construct with a detectable moiety that is not a radioisotope. For example, a mannosylated dextran construct could be labeled with a fluorescent moiety. Such a fluorescent construct can be used to localize to a tumor due to its interaction with and binding to CD206 on TAMs and MDSCs. The tumor localized fluorescent construct could then be visualized by appropriate excitation with detection and imaging by a human observer (seeing with their eyes) or by a camera capable of detecting the appropriate fluorescent emission wavelength. Embodiment described herein can comprise a composition that targets a CD206 expressing cell, wherein the composition labeled with a fluorescent moiety will bind to the CD206. In certain compositions, the composition can be used for detecting tumors. In some embodiments, a composition can be used to measure the size of a tumor. In certain embodiments, a composition can be used to deliver a therapeutic agent to a tumor and/or cancer cells.

Certain embodiments comprise a compound comprising a dextran backbone having one or more CD206 targeting moieties and one or more diagnostic moieties attached thereto. Embodiments can comprise a compound according to claim 1, wherein the compound is a compound of Formula (II):

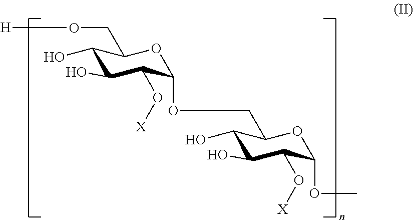

Wherein n is 1 or more and each X is independently H, L₁-A, or L₂-R; each L₁ and L₂ are independently linkers; each A independently comprises a detection moiety or H; each R independently comprises a CD206 targeting moiety or H; and wherein at least one R is a CD206 targeting moiety and at least one A is a diagnostic moiety. In some embodiments, at least one R is selected from the group consisting of mannose, fucose, and n-acetylglucosamine. In some embodiments, at least one A is a gamma-emitting agent. In some embodiments, at least one A can be selected from the group consisting of ^{99m}Tc, ¹¹¹In, and ¹²³I. In some embodiments, the at least one A can be an isotope. In some embodiments, the at least one A is selected from the group consisting of ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr. In some embodiments, at least one L₁ a C₂₋₁₂ hydrocarbon chain optionally interrupted by up to three heteroatoms selected from the group consisting of O, S and N. In some embodiments, at least one L₁ comprises —(CH₂)ₚS(CH₂)_{q}NH—, wherein p and q are integers from 1 to 5. In some embodiments, at least one L₂ is a C₂₋₁₂ hydrocarbon chain optionally interrupted by up to nine heteroatoms selected from the group consisting of O, S and N. In some embodiments, at least one L₂ comprises —(CH₂)ₚS(CH₂)_{q}NH—, wherein p and q independently are integers from 1 to 5. In certain embodiments, the at least one A is a contrast agent suitable for computed tomographic (CT) imaging and the at least one A is selected from the group consisting of iodinated molecules, ytterbium and dysprosium.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer Corporation, U.S.A.).

As used in the specification, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Unless otherwise indicated, references in the specification to parts by weight of a particular element or component in a composition denotes the weight relationship between the element or component and any other elements or components in the composition or article for which a part by weight is expressed, unless expressly described otherwise. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the identification can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent.

The term “contacting” as used herein refers to bringing a disclosed compound and a cell, a target receptor (e.g. CD206, or other receptor), or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, The specific effective amount for any particular subject will depend upon a variety of factors, including the disorder being diagnosed and the severity of the disorder; the specific composition employed; the age, body weight, general health, sex and diet of the patient; the time of administration; the route of administration; the rate of excretion of the specific compound employed; the duration of the diagnosis; drugs used in combination or coincidental with the specific compound employed and like factors well known in the medical arts. For example, it is well within the skill of the art to start doses of a compound at levels lower than those required to achieve the desired diagnostic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein the term “non-invasive” can refer to techniques that do not include the insertion or introduction of any instruments into a subject. For instance, administration of a diagnostic agent with a diagnostic moiety could be injected into a subject as described herein and then imaging, measuring, analyzing techniques described herein can be used to exercise the methods described herein. The term “non-invasive” will be clear to the skilled artisan when viewing the term in the context in which it used herein.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

“Alkyl” refers to a saturated aliphatic hydrocarbon including straight chain and branched chain groups. “Alkyl” may be exemplified by groups such as methyl, ethyl, n-propyl, isopropyl, n-butyl and the like. Alkyl groups may be substituted or unsubstituted. More than one substituent may be present. Substituents may also be themselves substituted. When substituted, the substituent group is preferably but not limited to C₁-C₄ alkyl, aryl, heteroaryl, amino, imino, cyano, halogen, alkoxy or hydroxyl. “C₁-C₄ alkyl” refers to alkyl groups containing one to four carbon atoms.

“Alkenyl” refers to an unsaturated aliphatic hydrocarbon moiety, including straight chain and branched chain groups. Alkenyl moieties must contain at least one alkene. “Alkenyl” may be exemplified by groups such as ethenyl, n-propenyl, isopropenyl, n-butenyl and the like. Alkenyl groups may be substituted or unsubstituted. More than one substituent may be present. When substituted, the substituent group is preferably alkyl, halogen or alkoxy. Substituents may also be themselves substituted. Substituents can be placed on the alkene itself and also on the adjacent member atoms or the alkenyl moiety. “C₂-C₄ alkenyl” refers to alkenyl groups containing two to four carbon atoms.

“Alkynyl” refers to an unsaturated aliphatic hydrocarbon moiety including straight chain and branched chain groups. Alkynyl moieties must contain at least one alkyne. “Alkynyl” may be exemplified by groups such as ethynyl, propynyl, n-butynyl and the like. Alkynyl groups may be substituted or unsubstituted. More than one substituent may be present. When substituted, the substituent group is preferably alkyl, amino, cyano, halogen, alkoxyl or hydroxyl. Substituents may also be themselves substituted. Substituents are not on the alkyne itself but on the adjacent member atoms of the alkynyl moiety. “C₂-C₄ alkynyl” refers to alkynyl groups containing two to four carbon atoms.

“Acyl” or “carbonyl” refers to the group —C(O)R wherein R is alkyl; alkenyl; alkynyl, aryl, heteroaryl, carbocyclic, heterocarbocyclic; C₁-C₄ alkyl aryl or C₁-C₄ alkyl heteroaryl. C₁-C₄ alkylcarbonyl refers to a group wherein the carbonyl moiety is preceded by an alkyl chain of 1-4 carbon atoms.

“Alkoxy” refers to the group —O—R wherein R is acyl, alkyl alkenyl, alkyl alkynyl, aryl, carbocyclic; heterocarbocyclic; heteroaryl, C₁-C₄ alkyl aryl or C₁-C₄ alkyl heteroaryl.

“Amino” refers to the group —NR′R′ wherein each R′ is, independently, hydrogen, amino, hydroxyl, alkoxyl, alkyl, aryl, cycloalkyl, heterocycloalkyl, heteroaryl, C₁-C₄ alkyl aryl or C₁-C₄ alkyl heteroaryl. The two R′ groups may themselves be linked to form a ring. The R′ groups may themselves be further substituted, in which case the group also known as guanidinyl is specifically contemplated under the term ‘amino”.

“Aryl” refers to an aromatic carbocyclic group. “Aryl” may be exemplified by phenyl. The aryl group may be substituted or unsubstituted. More than one substituent may be present. Substituents may also be themselves substituted. When substituted, the substituent group is preferably but not limited to heteroaryl, acyl, carboxyl, carbonylamino, nitro, amino, cyano, halogen, or hydroxyl.

“Carboxyl” refers to the group —C(═O)O—C₁-C₄ alkyl.

“Carbonyl” refers to the group —C(O)R wherein each R is, independently, hydrogen, alkyl, aryl, cycloalkyl; heterocycloalkyl, heteroaryl, C₁-C₄ alkyl aryl or C₁-C₄ alkyl heteroaryl.

“Carbonylamino” refers to the group —C(O)NR′R′ wherein each R′ is, independently, hydrogen, alkyl, aryl, cycloalkyl, heterocycloalkyl, heteroaryl, C₁-C₄ alkyl aryl or C₁-C₄ alkyl heteroaryl. The two R′ groups may themselves be linked to form a ring.

“C₁-C₄ alkyl aryl” refers to C₁-C₄ alkyl groups having an aryl substituent such that the aryl substituent is bonded through an alkyl group. “C₁-C₄ alkyl aryl” may be exemplified by benzyl.

“C₁-C₄ alkyl heteroaryl” refers to C₁-C₄ alkyl groups having a heteroaryl substituent such that the heteroaryl substituent is bonded through an alkyl group.

“Carbocyclic group” or “cycloalkyl” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the ring. Carbocyclic groups may be substituted or unsubstituted. More than one substituent may be present. Substituents may also themselves be substituted. Preferred carbocyclic groups include cyclopropyl, cyclobutyl, cyclopentyl, cyclohexyl, cyclohexenyl, and cycloheptyl. More preferred carbocyclic groups include cyclopropyl and cyclobutyl. The most preferred carbocyclic group is cyclopropyl. Carbocyclic groups are not aromatic.

As also used herein, the term “diagnosing” means determining the presence or absence of a medical condition, as well as determining or confirming the status of a previously confirmed medical condition in a patient. For example, in the case of cancer, the term diagnosing encompasses determining the presence or absence of cancer, the stage of cancer, and/or the detection of the presence, absence, or stage of a precancerous condition in a patient. Determining the status of a previously confirmed medical condition also includes determining the progress, lack of progress, decline or remission of a medical condition (e.g., a macrophage-related disorder).

“Halogen” refers to fluoro, chloro, bromo or iodo moieties. Preferably, the halogen is fluoro, chloro, or bromo.

“Heteroaryl” or “heteroaromatic” refers to a monocyclic or bicyclic aromatic carbocyclic radical having one or more heteroatoms in the carbocyclic ring. Heteroaryl may be substituted or unsubstituted. More than one substituent may be present. When substituted, the substituents may themselves be substituted. Preferred but non limiting substituents are aryl, C₁-C₄ alkylaryl, amino, halogen, hydroxy, cyano, nitro, carboxyl, carbonylamino, or C₁-C₄ alkyl. Preferred heteroaromatic groups include tetrazoyl, triazolyl, thienyl, thiazolyl, purinyl, pyrimidyl, pyridyl, and furanyl. More preferred heteroaromatic groups include benzothiofuranyl; thienyl, furanyl, tetrazoyl, triazolyl, and pyridyl.

“Heteroatom” means an atom other than carbon in the ring of a heterocyclic group or a heteroaromatic group or the chain of a heterogeneous group. Preferably, heteroatoms are selected from the group consisting of nitrogen, sulfur, and oxygen atoms. Groups containing more than one heteroatom may contain different heteroatoms.

“Heterocarbocyclic group” or “heterocycloalkyl” or “heterocyclic” means a monovalent saturated or unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, preferably 4 to 7 carbon atoms, and more preferably 5 to 6 carbon atoms in the ring. Bicyclic heterocarbocyclic groups contain 8 to 12 carbon atoms, preferably 9 to 10 carbon atoms in the ring. Heterocarbocyclic groups may be substituted or unsubstituted. More than one substituent may be present. Substituents may also be themselves substituted. Preferred heterocarbocyclic groups include epoxy, tetrahydrofuranyl, azacyclopentyl, azacyclohexyl, piperidyl, and homopiperidyl. More preferred heterocarbocyclic groups include piperidyl, and homopiperidyl. The most preferred heterocarbocyclic group is piperidyl. Heterocarbocyclic groups are not aromatic.

“Hydroxy” or “hydroxyl” means a chemical entity that consists of —OH. Alcohols contain hydroxy groups. Hydroxy groups may be free or protected. An alternative name for hydroxy is hydroxyl.

“Leash/leashes” and “linker/linkers” may be used interchangeably herein. The term “leash” or “leashes” can often be used to refer to attachment moiety used for a targeting moiety, such as mannose. The term “linker” or “linkers” can be used to refer to the attachment moiety used for a diagnostic and/or therapeutic moiety that may incorporate additional properties related to the chemistry of the linker and diagnostic and/or therapeutic moiety and the delivery of the said agent. Although these terms can be used interchangeably herein, their meaning will be clear to the skilled artisan in view of the context with which it is used.

“Member atom” means a carbon, nitrogen, oxygen or sulfur atom. Member atoms may be substituted up to their normal valence. If substitution is not specified the substituents required for valency are hydrogen.

“Ring” means a collection of member atoms that are cyclic. Rings may be carbocyclic, aromatic, or heterocyclic or heteroaromatic, and may be substituted or unsubstituted, and may be saturated or unsaturated. More than one substituent may be present. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Thioalkyl” refers to the group —S-alkyl.

“Tilmanocept” can refer to a non-radiolabeled precursor of the LYMPHOSEEK® compound. Compositions described herein may be a mannosylaminodextran. They can have a dextran backbone to which a plurality of amino-terminated linkers (—O(CH₂)₃S(CH₂)₂NH₂) are attached to the core glucose elements. In addition, mannose moieties can be conjugated to amino groups of a number of the linkers, and the chelator diethylenetriamine pentaacetic acid (DTPA) can be conjugated to the amino group of other linkers not containing the mannose. Compositions described herein can have a dextran backbone, in which a plurality of glucose residues comprise an amino-terminated linker:

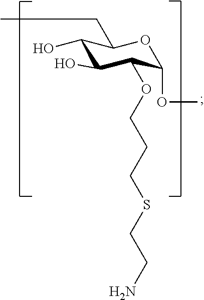

The mannose moieties can be conjugated to the amino groups of the linker via an amidine linker:

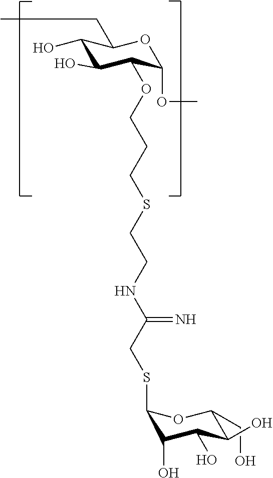

The chelator diethylenetriamine pentaacetic acid (DTPA) can be conjugated to the amino groups the linker via an amide linker:

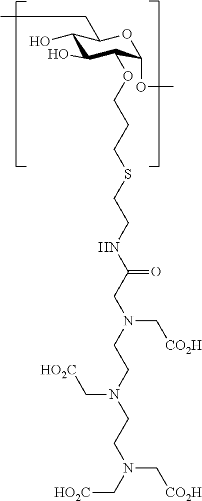

As described in the prescribing information approved for LYMPHOSEEK® in the United States, tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, has the following molecular formula: [C₆H₁₀O₅]ₙ●(C₁₉H₂₈N₄O₉S^{99m}Tc)_{b}●(C₁₃H₂₄N₂O₅S₂)_{c}●(C₅H₁₁NS)ₐ, and contains 3-8 conjugated DTPA molecules; 12-20 conjugated mannose molecules; and 0-17 amine side chains remaining free. Tilmanocept has the following general structure:

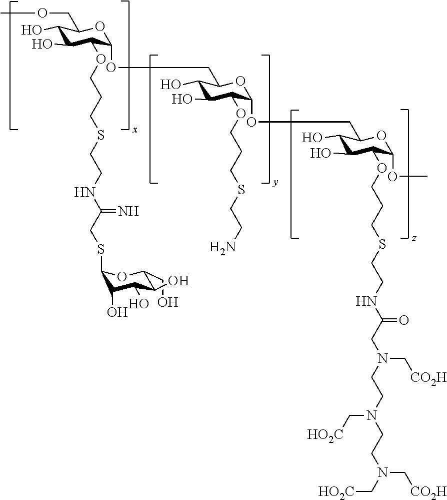

Certain of the glucose moieties may have no attached amino-terminated linker.

“Sulfonyl” refers to the —S(O)₂R′ group wherein R′ is alkoxy, alkyl, aryl, carbocyclic, heterocarbocyclic; heteroaryl, C₁-C₄ alkyl aryl or C₁-C₄ alkyl heteroaryl.

“Sulfonylamino” refers to the —S(O)₂NR′R′ group wherein each R′ is independently alkyl, aryl, heteroaryl, C₁-C₄ alkyl aryl or C₁-C₄ alkyl heteroaryl.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein can comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹³N, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes may be used for their ease of preparation and detectability. Further, substitution with heavier isotopes such as deuterium, i.e., ²H, can afford certain advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VC H Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules, including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Compounds

Embodiments of the present invention can employ a carrier construct comprising a polymeric (e.g., carbohydrate) backbone that can comprise a CD206 targeting moiety attached thereto (e.g., mannose) to deliver one or more active pharmaceutical ingredients. Examples of such constructs include mannosylamino dextrans (MAD), which can comprise a dextran backbone having conjugated to glucose residues of the backbone mannose molecules and having conjugated to other glucose residues of the backbone an active pharmaceutical ingredient. Tilmanocept is a specific example of a MAD. A tilmanocept derivative that is tilmanocept without DTPA conjugated thereto is a further example of a MAD (sometimes referred to as m-tilmanocept).

In some embodiments, the present invention provides a compound comprising a dextran-based moiety or backbone having one or more CD206 targeting moieties. The dextran-based moiety generally comprises a dextran backbone similar to that described in U.S. Pat. No. 6,409,990 (the '990 patent), which is incorporated herein by reference in its entirety. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, CD206 targeting moieties are attached to between about 10% and about 50% of the glucose residues of the dextran backbone, or between about 20% and about 45% of the glucose residues, or between about 25% and about 40% of the glucose residues. In some embodiments, every three glucose residues may be substituted. In some embodiments, every four glucose residues may be substituted. In some embodiments, every five glucose residues may be substituted. Some embodiments may comprise one mannose positioned on every third glucose residue. Some embodiments may comprise one mannose positioned on every fourth glucose residue. Some embodiments may comprise one mannose positioned on every fifth glucose residue. In some embodiments, the dextran-based moiety is about 50-100 kilodaltons (kDa). The dextran-based moiety may be at least about 50 kDa, at least about 60 kDa, at least about 70 kDa, at least about 80 kDa, or at least about 90 kDa. The dextran-based moiety may be less than about 100 kDa, less than about 90 kDa, less than about 80 kDa, less than about 70 kDa, or less than about 60 kDa. In some embodiments, the dextran backbone has a molecular weight (MW) of between about 1 and about 50 kDa, while in other embodiments the dextran backbone can have a MW of between about 5 and about 25 kDa. In embodiments, the dextran backbone can have a MW of between about 8 and about 15 kDa, such as about 10 kDa. While in other embodiments the dextran backbone can have a MW of between about 1 and about 5 kDa, such as about 2 kDa. Certain embodiments of compositions can comprise a backbone that is between about 1 to about 5 kDa, about 1 to about 10 kDa, about 1 to about 15 kDa, about 5 to about 12 kDa, about 5 to about 10 kDa, and ranges therebetween. In some embodiments, a composition may comprise between about 3 to about 7 mannose molecules, about 5 to about 10 mannose molecules, about 10 to about 15 mannose molecules, about 15 to about 20 mannose molecules, about 16 to about 17 mannose molecules, and ranges therebetween. In some embodiments, a backbone may be about 1 to about 3 kDa and may further comprise about 3 to about 7 mannose molecules. In some embodiments, a backbone may be about 10 kDa and may further comprise about 15 to about to about 20, or about 16 to about 17 mannose molecules. An embodiment may comprise a backbone that is about 10 kDa and further comprise about 16 to about 17 mannose molecules. Such a configuration has unexpectedly superior and improved solubility, improved clarity, improved injectability and distribution.

Some embodiments may comprise a backbone that is not a dextran backbone. Some embodiments may have a monosaccharide-based backbone that does not comprise dextran. The backbone of a carbohydrate-based carrier molecules described herein can comprise a glycan other than dextran, wherein the glycan comprises a plurality of monosaccharide residues (i.e., sugar residues or modified sugar residues). In certain embodiments, the glycan backbone has sufficient monosaccharide residues, as well as optional groups such as one or more amino acids, polypeptides and/or lipids, to provide a MW of about 1 to about 50 kDa. The glycan can comprise oligosaccharides or polysaccharides. As would be appreciated by the skilled artisan when considering the disclosure contained herein, when referring to a “dextran” backbone, other monosaccharide residues may be considered to be substituted in compounds described herein. Additional descriptions of carbohydrate-backbone-based carrier molecules used for targeting CD206 are described in PCT application No. US/2017/055211, which is herein incorporated by reference in its entirety.

In any of the embodiments where the backbone is conjugated with one or more primary carbohydrates (monosaccharides), such carbohydrates can comprise any of a variety of sugar and modified sugar residues (e.g., sulfated, brominated, or nitrogenated sugar residues), including one or more of: fucose, arabinose, allose, altrose, glucose, galactose, glucose, galactosamine, n-acetylgalactosamine, hammelose, lyxose, levoglucosenone, mannose, mannitol, mannosamine, n-acetylmannosamine, ribose, rhamnose, threose, talose, xylose and combinations of two or more of the foregoing. In certain embodiments, a backbone of compositions herein may comprise a carbohydrate moiety that does not comprise glucose and may be any suitable polymer. These moieties may include, for example but without limitation, fucose, n-acetylglucoseamine, n-acetylgalactoseamine, galactose, neuraminate, and the like. The backbone may be heterogeneous, containing more than one species of sugar and/or carbohydrate.

The carrier molecules used in the compositions, kits, diagnostic, and therapeutic methods described herein are used to deliver a diagnostic and/or therapeutic moiety (e.g., a cytotoxic agent). The carrier molecules include one or more features which allow a detectable moiety to be attached to the molecule, either directly or indirectly (e.g., using a leash). In some embodiments, the carbohydrate-based backbone has a MW of between about 1 and about 50 kDa, while in other embodiments the carbohydrate-based backbone has a MW of between about 5 and about 25 kDa. In still other embodiments, the carbohydrate-based backbone has a MW of between about 8 and about 15 kDa, such as about 10 kDa. While in other embodiments the carbohydrate-based backbone has a MW of between about 1 and about 5 kDa, such as about 2 kDa. The MW of the carbohydrate-based backbone may be selected based upon the inflammasome-mediated disorder. In addition, unlike the dextran backbone of the '990 patent, the carbohydrate-based backbones described herein do not necessarily need to be crosslink-free, and larger MW backbones (>50 kDa) may be employed in some instances.

Any of a variety of detectable moieties can be attached to the carrier molecule, directly or indirectly, for a variety of purposes. As used herein, the term “detectable moiety” or “diagnostic moiety” (which these terms may be used interchangeably) means an atom, isotope, or chemical structure which is: (1) capable of attachment to the carrier molecule; (2) non-toxic to humans; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means as well as by the measurement processes described herein.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents.

By way of specific example, suitable diagnostic moieties include, but are not limited to:
contrast agents suitable for magnetic resonance imaging (MRI), such as gadolinium (Gd³⁺), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr; or other chelateable isotope(s);
gamma-emitting agents suitable for single-photon emission computed tomography (SPECT), such as ^{99m}Tc, ¹¹¹In, and ¹²³I.
dyes and fluorescent agents suitable for optical imaging
agents suitable for positron emission tomography (PET) such as ¹⁸F, ¹³N, ⁶⁴Cu, ²²³Rb.

A diagnostic or therapeutic moiety can be attached to the carrier molecule in a variety of ways, such as by direct attachment or using a chelator attached to a carrier molecule. In some embodiments, diagnostic or therapeutic moieties can be attached using leashes attached to a carrier backbone. Thereafter, and as described in the ties as by direct attack can be conjugated to an amino group of one or more leashes and can be used to bind the diagnostic or therapeutic moiety thereto. It should be noted that in some instances, glucose moieties may have no attached aminothiol leash. Certain embodiments may include a single type of diagnostic or therapeutic moiety or a mixture of different diagnostic and/or therapeutic moieties. For example, an embodiment of a compound disclosed herein may comprise a contrast agent suitable for MRI and a radioisotope suitable for scintigraphic imaging, and further combinations of the diagnostic and/or therapeutic moieties described herein. In some embodiments, gallium (e.g., ⁶⁸Ga) may be preferred because of superior results of image resolution.

One or more diagnostic or therapeutic moieties can be attached to the one or more leashes using a suitable chelator. Suitable chelators include ones known to those skilled in the art or hereafter developed, such as, for example but without limitation, tetraazacyclododecanetetraacetic acid (DOTA), mercaptoacetylglycylglycyl-glycine (MAG3), diethylenetriamine pentaacetic acid (DTPA), dimercaptosuccinic acid, diphenylehtylene diamine, porphyrin, iminodiacetic acid, and ethylenediaminetetraacetic acid (EDTA).

Certain embodiments of compositions can comprise a backbone that is between about 1 to about 5 kDa, about 1 to about 10 kDa, about 1 to about 15 kDa, about 5 to about 12 kDa, about 5 to about 10 kDa, and ranges there between. In some embodiments, a composition may comprise between about 2 to about 7 mannose molecules, about 5 to about 10 mannose molecules, about 10 to about 15 mannose molecules, about 15 to about 28 mannose molecules, about 16 to about 17 mannose molecules, and ranges there between. In some embodiments, a backbone may be about 1 to about 3 kDa and may further comprise about 3 to about 7 mannose molecules. In some embodiments, a backbone may be about 10 kDa and may further comprise about 15 to about to about 20, or about 16 to about 17 mannose molecules.

In some embodiments, the CD206 targeting moiety is selected from, but not limited to, mannose, fucose, fucoid, galactose, n-acetylgalactosamine, and n-acetylglucosamine and combinations of these. In some embodiments, the targeting moieties are attached to between about 10% and about 50% of the glucose residues of the dextran backbone, or between about 20% and about 45% of the glucose residues, or between about 25% and about 40% of the glucose residues. (It should be noted that the MWs referenced herein, as well as the number and degree of conjugation of receptor substrates, linkers, and diagnostic moieties attached to the dextran backbone refer to average amounts for a given quantity of carrier molecules, since the synthesis techniques will result in some variability.)

In some embodiments, the one or more CD206 targeting moieties and one or more detection labels are attached to the dextran-based moiety through a linker. The linker may be attached at from about 50% to about 100% of the backbone moieties or about 70% to about 90%. In embodiments with multiple linkers, the linkers may be the same or different. In some embodiments, the linker is an amino-terminated linker. In some embodiments, the linkers may comprise —O(CH₂)₃S(CH₂)₂NH—. In some embodiments, the linker may be a chain of from 1 to 20 member atoms selected from carbon, oxygen, sulfur, nitrogen and phosphorus. The linker may be a straight chain or branched. The linker may also be substituted with one or more substituents including, but not limited to, halo groups, perfluoroalkyl groups, perfluoroalkoxy groups, alkyl groups, such C₁₋₄ alkyl, alkenyl groups, such as C₁₋₄ alkenyl, alkynyl groups, such as C₁₋₄ alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkyl sulfonyl groups, arylsulfonyl groups, —NH—NH₂; ═N—H; ═N-alkyl; —SH; —S-alkyl; —NH—C(O)—; —NH—C(═N)— and the like. Other suitable linkers would be known to one of ordinary skill in the art.

In some embodiments, the one or more diagnostic and/or therapeutic moieties can be attached via a biodegradable linker. In some embodiments, the biodegradable linker comprises an acid sensitive, such as a hydrazone moiety. In certain embodiments, the linker comprises a biodegradable moiety attached to a linker.

In some embodiments, a carrier molecules used in therapeutic and diagnostic methods and compositions described herein can comprise a therapeutic agent attached to the carrier molecule—either in place of a detectable moiety or in conjunction therewith. As used herein, the term “therapeutic agent” means an atom, isotope, or chemical structure that is effective in curing or eliminating a disease or other condition, as well those which are effective in reducing, slowing the progress of, or ameliorating the adverse effects of a disease or other condition. Therapeutic agents can include cytotoxic agents.

In some embodiments, a therapeutic agent comprises a high energy killing isotope that has the ability to kill macrophages and tissue in the surrounding macrophage environment. Suitable radioisotopes include: ^{210/212/213/214}Bi, ^{131/140}Ba, _{11/14}C, ⁵¹Cr, ^{67/68}Ga, ¹⁵³Gd, ^{99m}Tc, ^{88/90/91}Y, ^{123/124/125/131}I, ^{111/115m}In, ¹⁸F, ¹³N, ¹⁰⁵Rh, ¹⁵³Sm, ⁶⁷Cu, ⁶⁴Cu, ¹⁶⁶Ho, ²²³Rb, ¹⁷⁷Lu, ¹⁸⁶Re and ¹⁸⁸Re, ^{32/33}P, ^{46/47}Sc, ^{72/75}Se, ³⁵S, ¹⁸²Ta, ^{123m/127/129/132}Te, ⁶⁵Zn and ^{89/95}Zr.

In some embodiments, a therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr.

In some embodiments, a therapeutic agent can be selected from the group consisting of cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs. By way of example, a therapeutic agent can be selected from the group consisting of mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, and dynemicins.

In embodiments where a therapeutic agent can be a hormone or hormone antagonist, the therapeutic agent may be selected from the group consisting of prednisone, hydroxyprogesterone, medroprogesterone, diethylstilbestrol, tamoxifen, testosterone, and aminogluthetimide.

In embodiments where a therapeutic agent is a prodrug, the therapeutic agent may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug.

A therapeutic agent can be attached to a carrier molecule in a variety of ways. In some embodiments, one or more leashes can be conjugated to a backbone molecule, and a chelator can be conjugated to one or more leashes (e.g., to the amino group of amino-terminated leashes). A chelator can be used to bind a therapeutic agent thereto. Suitable chelators include ones known to those skilled in the art or hereafter developed, such as, for example, tetraazacyclododecanetetraacetic acid (DOTA), mercaptoacetylglycylglycyl-glycine (MAG3), diethylenetriamine pentaacetic acid (DTPA), dimercaptosuccinic acid, diphenylehtylene diamine, porphyrin, iminodiacetic acid, and ethylenediaminetetraacetic acid (EDTA).

Macromolecular compounds described herein may be administered in a variety of ways, using any of a variety of pharmaceutically acceptable carriers and vehicles. For example, a pharmaceutical preparation comprising the carrier molecule having one or more detectable moieties and/or therapeutic agents attached thereto, in combination with a pharmaceutically acceptable carrier can be administered via intravenous injection, subcutaneous injection, intradermal injection, parenchymal introduction, inhalation, pulmonary lavage, suppository, or oral, sublingual, intracranial, intraocular, intranasal, or intraaural introduction.

In an embodiment for diagnosing and/or treating tuberculosis, the detectable moiety comprises ⁶⁸Ga, and the therapeutic agent comprises ⁶⁸Ga and/or Ga. In embodiments, a composition for both diagnosing and treating tuberculosis can be provided, wherein the both ⁶⁸Ga and Ga (i.e., non-radioactive Ga) are conjugated to the carrier molecule.

Various other linkers known to those skilled in the art or subsequently discovered may be used in place of (or in addition to) —O(CH₂)₃S(CH₂)₂NH₂. These include, for example, bifunctional linker groups such as alkylene diamines (H₂N—(CH₂)ᵣ—NH₂), where r is from 2 to 12; aminoalcohols (HO—(CH₂)ᵣ—NH₂), where r is from 2 to 12; aminothiols (HS—(CH₂)ᵣ—NH₂), where r is from 2 to 12; amino acids that are optionally carboxy-protected; ethylene and polyethylene glycols (H—(O—CH₂—CH₂)ₙ—OH, where n is 1-4). Suitable bifunctional diamines include ethylenediamine, 1,3-propanediamine, 1,4-butanediamine, spermidine, 2,4-diaminobutyric acid, lysine, 3,3′-diaminodipropylamine, diaminopropionic acid, N-(2-aminoethyl)-1,3-propanediamine, 2-(4-aminophenyl)ethylamine, and similar compounds. One or more amino acids also can be employed as the bifunctional linker molecule, such as β-alanine, γ-aminobutyric acid or cysteine, or an oligopeptide, such as di- or tri-alanine.

Other bifunctional linkers include:

—NH—(CH₂)ᵣ—NH—, where r is from 2-5,

—O—(CH₂)ᵣ—NH—, where r is from 2-5,

—NH—CH₂—C(O)—,

—O—CH₂—CH₂—O—CH₂—CH₂—O—,

NH—NH—C(O)—CH₂—,

—NH—C(CH₃)₂C(O)—,

—S—(CH₂)ᵣ—C(O)—, where r is from 1-5,

—S—(CH₂)ᵣ—NH—, where r is from 2-5,

—S—(CH₂)ᵣ—O—, where r is from 1-5,

—S—(CH₂)—CH(NH₂)—C(O)—,

—S—(CH₂)—CH(COOH)—NH—,

—O—CH₂—CH(OH)—CH₂—S—CH(CO₂H)—NH—,

—O—CH₂—CH(OH)—CH₂—S—CH(NH₂)—C(O)—,

—O—CH₂—CH(OH)—CH₂—S—CH₂—CH₂—NH—,

—S—CH₂—C(O)—NH—CH₂—CH₂—NH—, and

—NH—O—C(O)—CH₂—CH₂-O—P(O₂H)—.

Examples of constructs useful in the present invention include mannosylamino dextrans (MAD) such as tilmanocept and m-tilmanocept. In some embodiments, the dextran-based moiety having at least one CD206 targeting moiety attached thereto can be a compound of Formula (I):

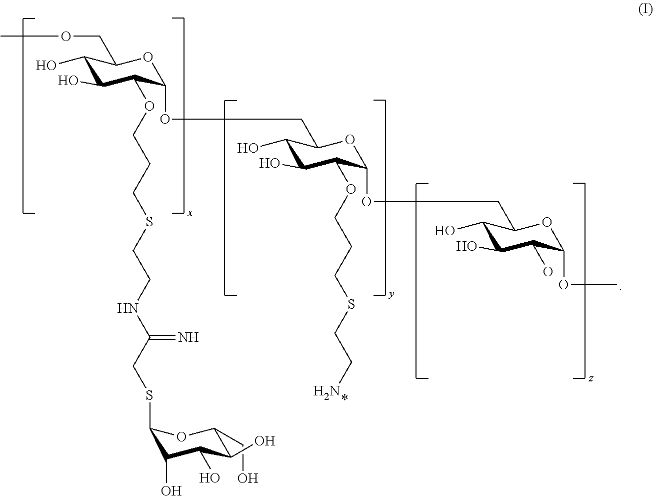

wherein the * indicates the point at which a diagnostic or therapeutic moiety can be attached. In certain embodiments, a diagnostic or therapeutic moiety can be attached via a linker. In certain embodiments, x can be between about 10 to about 25, about 5 to about 25, about 10 to about 20, about 15 to about 25, about 15 to about 20 and ranges therebetween. In some embodiments, y can be between about 35 and about 70, about 40 and about 70, about 50 and about 65, and ranges therebetween. In some embodiments, z can be between about 40 to about 70, about 50 to about 65, about 50 to about 60 and ranges therebetween.

In other embodiments, the compound of the present invention can be a compound of Formula (II):

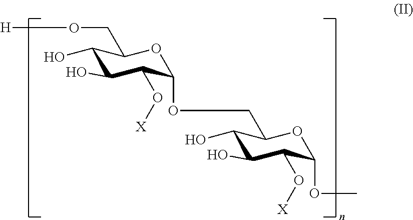

Wherein

each X is independently H, L₁-A, or L₂-R; each L₁ and L₂ are independently linkers;

each A independently comprises a detection label or H;

each R independently comprises a CD206 targeting moiety or H; and

n is an integer greater than zero.

In certain embodiments, L₁ is a linker as described above. In certain embodiments, L₂ is a linker as described above.

In some embodiments a dosage of a compound described herein can comprise between about 5-500 μg of the compound, between about 200-300 μg of the compound, between about 100-300 μg of the compound, between about 100-200 μg of the compound, about 50-400 μg of the compound, about 125-175 μg of the compound, about 150 μg of the compound and ranges therebetween. In certain embodiments, the amount of radiolabeling can be altered to affect the radioactivity of a dose. For example, the radioactivity of about 0.1-50mCi, about 0.5-10 mCi, about 10-50 mCi, about 10 mCi, about 5-25 mCi, about 1-15mCi, and ranges therebetween.

### Synthesis

The compounds of this invention can be prepared by employing reactions as shown in the disclosed schemes, in addition to other standard manipulations that are known in the literature, exemplified in the experimental sections or clear to one skilled in the art. The following examples are provided so that the invention might be more fully understood, are illustrative only, and should not be construed as limiting. For clarity, examples having fewer substituents can be shown where multiple substituents are allowed under the definitions disclosed herein.

It is contemplated that each disclosed method can further comprise additional steps, manipulations, and/or components. It is also contemplated that any one or more step, manipulation, and/or component can be optionally omitted from the invention. It is understood that a disclosed method can be used to provide the disclosed compounds. It is also understood that the products of the disclosed methods can be employed in the disclosed compositions, kits, and uses.

The compounds of the present invention may be synthesized by any number of ways known to one of ordinary skill in the art. For example, linker 2 can be synthesized by opening succinic anhydride ring by tert-butyl carbazate. The resulting carboxylic acid is converted to the corresponding N-hydroxy succinimide (NHS) ester using EDC coupling reagent. MAD is then functionalized with linker 2 by forming an amide linkage. Then, the Boc protecting group can be removed under dilute acidic condition (typically 30-40% trifluoroacetic acid in DMSO) to obtain 4. Dilute acidic condition is required to avoid any unwanted cleavage of the glycosidic linkage present in dextran backbone. The resulting functionalized MAD can be purified by size exclusion filtration.

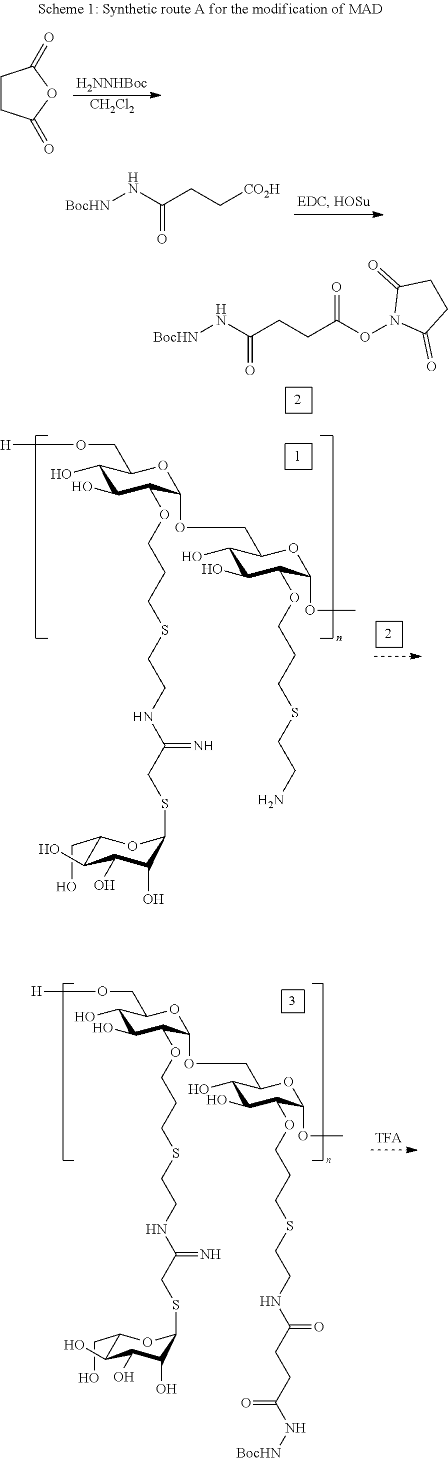

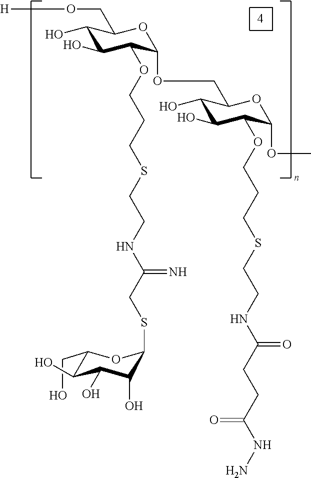

Alternatively, compounds according to the present invention may be synthesized according to Scheme 2. Free primary amine groups of MAD can be reacted with an excess of lactone under anhydrous condition. Unreacted lactone can be removed under reduced pressure to obtain modified MAD 6. The corresponding hydrazine derivative 7 can be prepared by reductive amination reaction using sodium cyanoborohydride or sodium triacetoxy borohydride as the reducing agent.

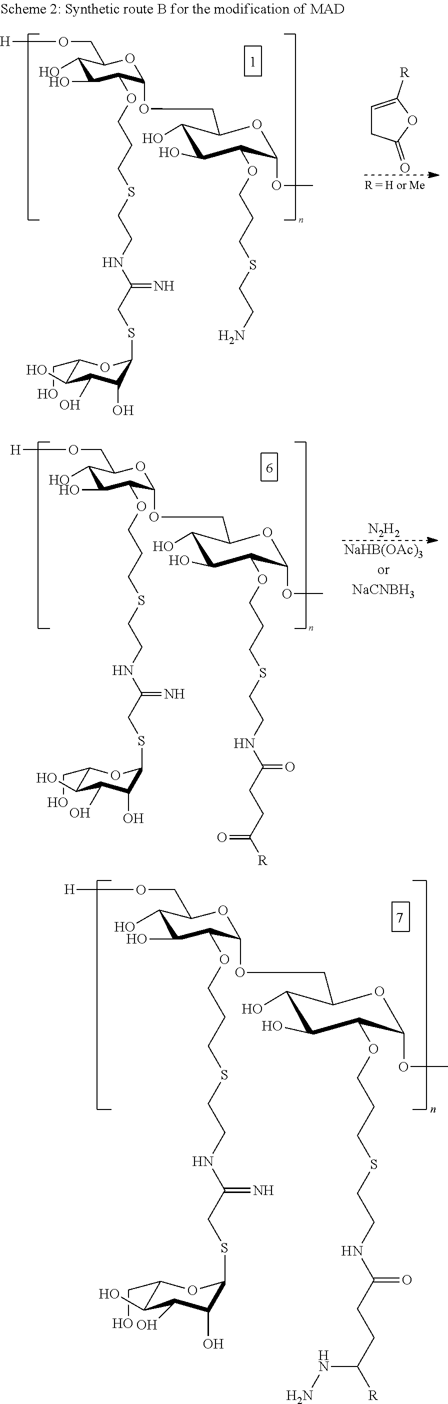

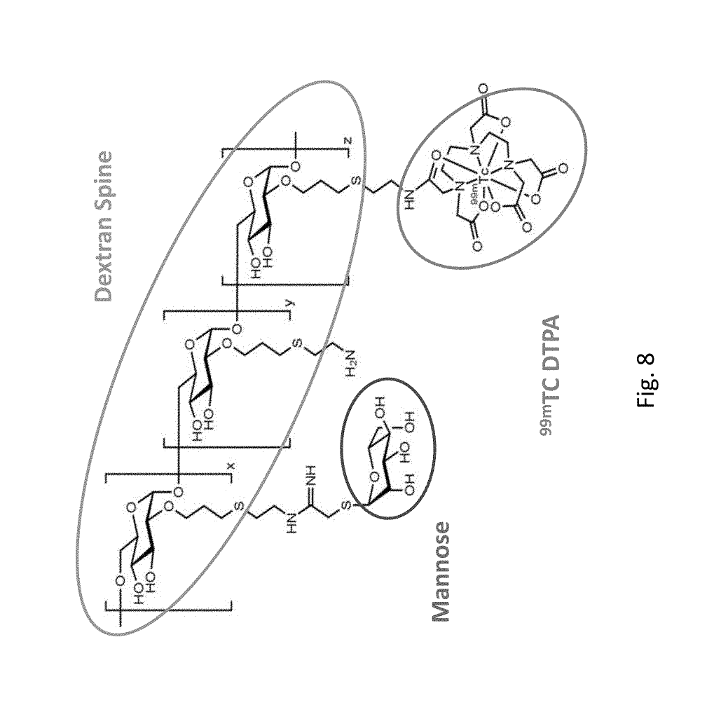

The conjugation of diagnostic and/or therapeutic moiety to MAD derivatives 4 or 7 can be as is shown in Scheme 3. MAD derivative 4 or 7 can be conjugated to a diagnostic or therapeutic moiety by formation of hydrazone linkage under anhydrous acidic condition or aqueous acidic conditions. An example of an embodiment of an composition as described herein is provided in FIG. 8.

One of ordinary skill in the art may recognize other ways to synthesize the compounds of the present invention in view of the present disclosure.

As used herein, mannosylated dextran molecular constructs (MDMCs) can be a class of compounds sharing the following characteristics:
1. A backbone comprised of dextran,
2. Molecular leashes attached to the glucose moieties of the dextran backbone,
3. Mannose sugars attached to a portion of the molecular leashes,
4. One or more diagnostic and/or therapeutic moieties attached to molecular leashes that are not occupied by mannose moieties,
5. Other sugar moieties in addition to mannose may optionally be added to molecular leashes that are not occupied by either mannose moieties or the diagnostic and/or therapeutic moieties.

Embodiments also can include mannosylated dextran constructs that are built on dextran backbones of varying sizes including but not limited to the 10 kilodalton dextran upon which tilmanocept is constructed. Mannosylated dextran constructs of differing sizes may exhibit differing performance characteristics with varying utilities. For example, smaller mannosylated constructs may penetrate into tumors more readily, providing for greater tumor localization and greater tumor specific signal (either radioactive or fluorescent). Smaller constructs may also be excreted more rapidly into the urine, which would be expected to shorten the plasma residence time of the construct. This shorter plasma residence time would be expected to reduce nonspecific background associated with tumor imaging. Alternatively, a mannosylated dextran built on a dextran backbone larger than 10 kilodaltons may have a longer plasma residence time than tilmanocept. A longer plasma residence time could increase the opportunity of the construct to enter and penetrate a tumor, resulting in a greater tumor specific signal even though the construct may penetrate tumors more slowly.

Embodiments may also include mannosylated dextran constructs with varying numbers of mannose moieties as long as the number of mannose moieties is sufficient to facilitate high affinity interactions with CD206 and/or tumor-associated macrophages. In addition, embodiments can include mannosylated dextran constructs in which the mannose moieties, DTPA and/or any other chemical moiety or moieties are attached to the dextran backbone by chemical leashes or linkers of any chemical composition including but not limited to the amine terminated leashes used in tilmanocept.

Compositions described herein may be used to detect primary tumors and metastases. This detection can be achieved through localization of mannosylated dextran constructs to tumor associated macrophages (TAMs), myeloid derived suppressor cells (MDSC) and other tumor associated immune cells that express CD206. In certain embodiments, the metastases may be liver metastases. In certain embodiments, a composition as described herein can be used to image a tumor. In some embodiments, the imaging is for a tumor that is not associated with breast cancer.

Certain embodiments described herein can comprise a composition for imaging a tumor, comprising: ^{99m}Tc-tilmanocept, wherein said composition is used for imaging a tumor.

Some embodiments comprise a composition for imaging one or more tumors, comprising: ^{99m}Tc-tilmanocept, wherein said composition is used for imaging one or more tumors.

Some embodiments comprise a mannosylated dextran molecular construct comprised of glucose moieties comprising: a backbone comprised of dextran, at least one leash attached to the glucose moieties of the dextran backbone, at least one mannose sugar attached to a portion of the at least one leash; and one or more detection moieties attached to the at least one leash. In certain embodiments, the at least one leash is not occupied by any mannose moieties. In some embodiments, the at least one other sugar moiety can be added to the at least one leash, wherein the at least one other sugar is not occupied by either mannose moiety, diagnostic, or therapeutic moiety.

Certain embodiments comprise a method of imaging a tumor comprising: administering a compound to a subject comprising a dextran backbone having one or more CD206 targeting moieties and one or more diagnostic moieties attached thereto; and imaging said subject using single-photon emission computed technology (SPECT) or positron-emission tomography (PET) with or without x-ray based computed technology (CT) (i.e., SPECT/CT or PET/CT) wherein an image comprises visual indications of uptake of said compound in one or tumors in a subject. In some embodiments, the imaging of a subject can be done using planar gamma imaging wherein an image comprises visual indications of uptake of said compound in one or more tumors in the subject. In certain embodiments, the method is for imaging tumors that are not a result of breast cancer. In certain embodiments, the composition further comprises a therapeutic agent.

Certain embodiments comprise a method of imaging a tumor comprising: administering a compound to a subject comprising a dextran backbone having one or more CD206 targeting moieties and one or more therapeutic moieties attached thereto; and imaging said subject using single-photon emission computed technology (SPECT) or positron-emission tomography (PET) with or without x-ray based computed technology (CT) (i.e., SPECT/CT or PET/CT) wherein an image comprises visual indications of uptake of said compound in one or tumors in a subject. In some embodiments, the imaging of a subject can be done using planar gamma imaging wherein an image comprises visual indications of uptake of said compound in one or more tumors in the subject. In certain embodiments, the method is for imaging tumors that are not a result of breast cancer.

In some embodiments, a tumor can be detected or imaged at any anatomical site. In certain embodiments, a detected and/or imaged tumor is a visceral tumor. In certain embodiments, a detected and/or imaged tumor is a metastatic tumor. In certain embodiments, a visceral tumor is a result of a cancer chosen from the group selected from lung, colorectal, renal, ovarian, prostate, testicular, pancreatic, lymphomas, and the like. In certain embodiments, a visceral tumor is a primary tumor. In certain embodiments, a metastatic tumor is a result of a cancer chosen from the group selected from lung, colorectal, renal, ovarian, prostate, testicular, pancreatic, lymphomas, and the like. In some embodiments, the metastatic tumor may occur in the same tissue type as a primary cancer (e.g., metastasis within the liver). In certain embodiments, the metastatic tumor is in a tissue that is different from a prior-existing primary cancer (e.g., the metastatic tumor may be detected in the liver whereas the subject may initially have one or more tumors associated with colorectal cancer).

Certain embodiments described herein comprise a composition for detecting one or more metastasized tumors, comprising: ^{99m}Tc-tilmanocept, wherein said composition is used for detecting one or more metastasized tumors. Certain embodiments can comprise a composition used for quantifying the size of a tumor, including estimating its mass and size.

Certain embodiments can comprise a composition for imaging tumor-associated macrophages. Certain embodiments can comprise a composition comprising a diagnostic moiety for imaging tumor-associated macrophages (TAMS). In certain embodiments, the compositions and methods described herein are not used for determining tumor margins. In certain embodiments, the compositions and methods described herein are not used intraoperatively. In certain embodiments, the compositions and methods described herein are not used to target dendritic cells.

Certain embodiments can comprise a composition comprising a mannosylated dextran construct. Certain embodiments can comprise a composition comprising a dextran backbone having one or more CD206 targeting moieties and one or more diagnostic moieties attached thereto. Certain embodiments can comprise a composition comprising a dextran backbone having one or more CD206 targeting moieties and one or more therapeutic moieties attached thereto. Certain methods can comprise the steps of administering a composition comprising a dextran backbone having one or more CD206 targeting moieties and one or more diagnostic moieties to a subject; imaging the subject using SPECT/CT; and determining the presence of tumor-associated macrophages. Certain embodiments may further comprise the step of quantifying the number of tumor-associated macrophages. Certain embodiments comprise the step of identifying the presence of tumors. Certain embodiments comprise the step of identifying metastases of previously-existing tumors. Certain embodiments comprise the step of administering a composition as described herein comprising a therapeutic agent for administering a therapeutic agent to a tumor. Certain embodiments comprise the step of administering a composition as described herein comprising a therapeutic agent for administering a therapeutic agent to a metastasized tumor.

### EXAMPLES

### Example 1

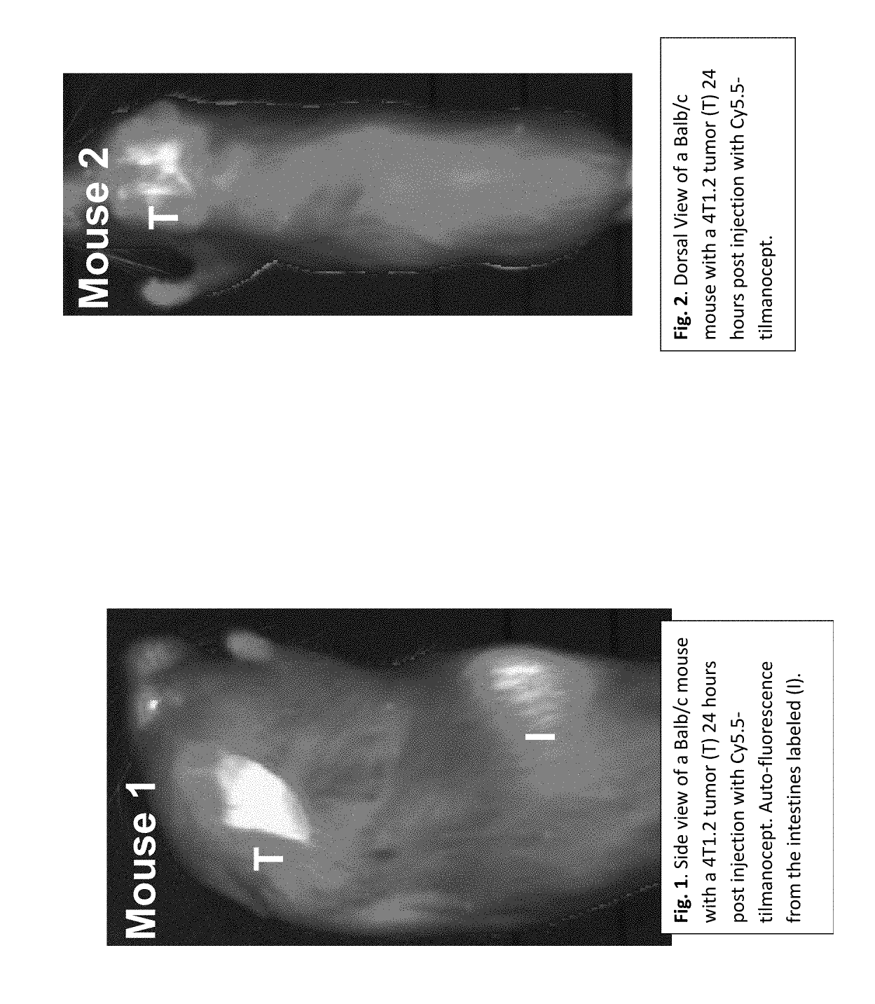

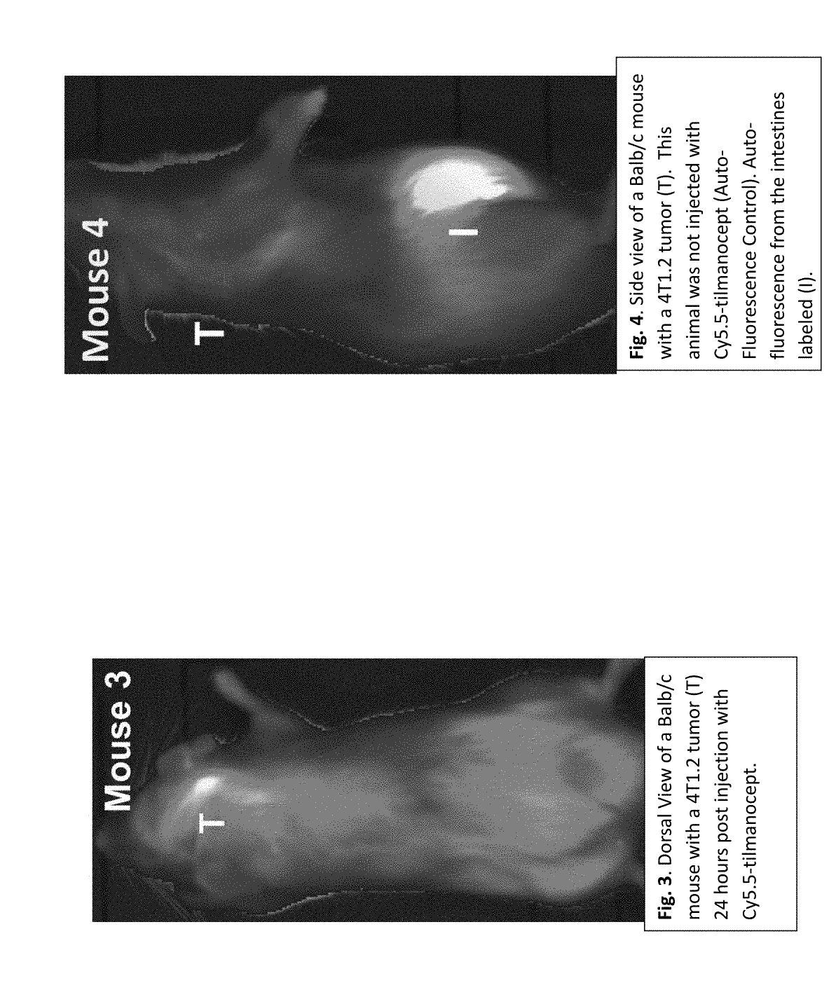

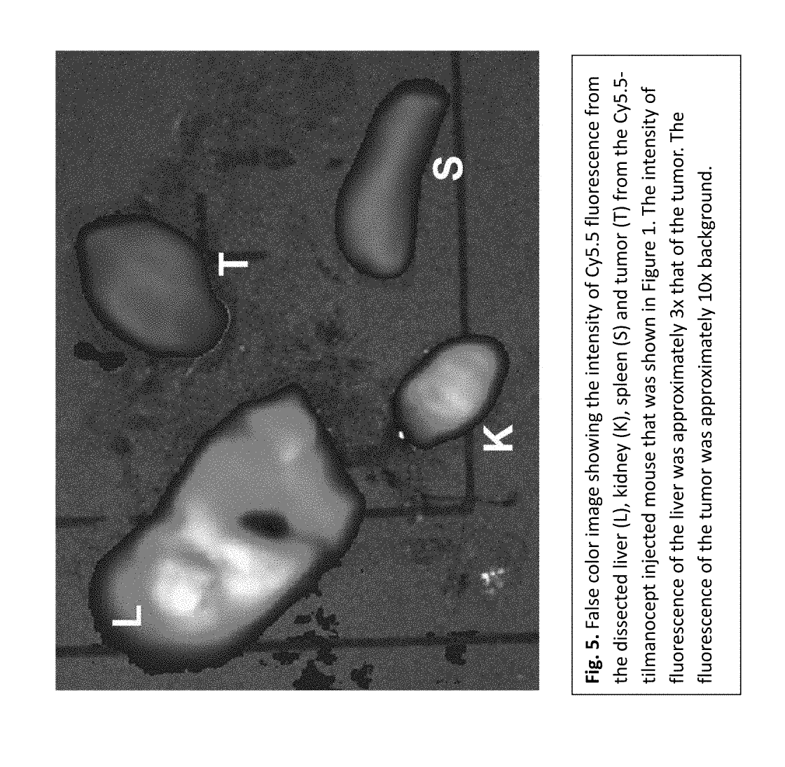

In the first experiment, 4T1.2/Balb-c syngeneic mouse model of breast cancer was evaluated for the specific localization of tilmanocept label with the fluorescent moiety, Cy5.5. Per protocol, four 6-8 week old female Balb/c mice were inoculated with culture propagated 4T1 cells by subdermal injection in their upper backs between their scapulae. When tumors reached between about 0.5-1.0 cm in diameter, 3 mice were injected intravenously into their tail veins with 4 μg (0.2 nM) of tilmanocept labeled with Cyanine 5.5 (Cy5.5). There were approximately 1.5 Cy5.5 moieties per tilmanocept molecule. Twenty-four hours post injection, the animals were humanely euthanized and evaluated for Cy5.5 specific fluorescence. One animal that hosted a 4T1.2 tumor but was not injected with Cy5.5-tilmanocept was also humanely euthanized and evaluated for autofluorescence and served as a control for autofluorescence. Animals were examined for fluorescence under exposure to a 640 nm excitation source. Images of the animals are shown in FIG. 1 through FIG. 4. Whole animal images were obtained after which the mice were necropsied to expose the individual organs, which were then imaged separately.

The animal shown in FIG. 4 was not injected with Cy5.5-tilmanocept and acted as the auto-fluorescence control. This image shows auto-fluorescence coming from the animal's fur and also from its abdomen. Necropsy of the animals revealed that the observed fluorescence from the abdomen was the result of auto-fluorescence from the intestines, possibly a consequence of the formulation of their diet chow. All four animals showed autofluorescence of their fur and intestines, which is unrelated to their exposure to Cy5.5-tilmanocept. The tumor on the back of the animal not injected with Cy5.5-tilmanocept did not demonstrate auto-fluorescence. The image of animal 4 (no Cy5.5-tilmanocept) differs significantly from the images of the other 3 mice in that in all three of the mice injected with Cy5.5-tilmanocept, Cy5.5 mediated fluorescence was observed through the skin and fur of all tumors from the mice injected with Cy5.5-tilmanocept, clearly demonstrating the detectable localization of Cy5.5-tilmanocept to tumors. FIGS. 1-4 are false color representations of the Cy5.5 fluorescence observed in the mice wherein yellow is approximately 3× more intense than the auto-fluorescence of the fur (red).

The mice that had been injected with Cy5.5-tilmanocept were dissected and their organs and tumors were evaluated for Cy5.5 mediated fluorescence. It is known from previously completed biodistribution studies conducted in wild-type healthy rats that have been injected intravenously with ^{99m}Tc-tilmanocept that significant portions of the injected doses quickly localize to the liver, kidneys and spleen, organs in which large numbers of CD206 expressing cells are known to normally reside. Most of the injected 99mTc-tilmanocept that does not localize to these organs is excreted into the urine. In these studies conducted in rats, there was a much higher localization of 99mTc-tilmanocept to the livers than to any other organ with more localizing to the kidneys than to the spleens. In all 3 mice injected with Cy5.5-tilmanocept and consistent with the biodistribution results from rats, there was significant localization of Cy5.5-tilmanocept to the livers, kidneys and spleens with the livers showing the highest density of localization followed by the kidneys then the spleens. In FIG. 5, the fluorescence of these three organs from mouse #1 (FIG. 1) are compared with mouse #1's dissected tumor. The fluorescence from the tumor was comparable to the fluorescent emanating from the spleen. Similar results were observed for the organs and tumors removed from the other two mice that had been injected with Cy5.5-tilmanocept. Importantly, measurements of the auto-fluorescence of the liver, kidneys, spleen and tumor of the forth mouse that was not injected with Cy5.5-tilmanocept showed that the levels of fluorescence observed in the organs and tumors of the Cy5.5-tilmanocept injected mice were approximately an order of magnitude greater than the auto-fluorescence observed for these tissues from the uninjected mouse.

In certain embodiments, the compositions and methods can be used to image tumors. Certain embodiments may be used to image non-malignant tumor cells. In certain embodiments, the compositions and methods used herein are not using PET scanning. Certain embodiments may use SPECT imaging. Some embodiments as described herein exclude the use of any antibody fragments for any imaging purpose as described herein. In some embodiments, a patient can receive multiple injections within 24 hours. In certain embodiments, a patient may receive several injections without experiencing any allergic reaction. Some embodiments described herein may be used to predict the performance of an immunotherapy regime. In some embodiments, CD206 TAMS density in a tumor may be used to predict a patient's response to a particular immunotherapy.

### Example 2

Two colorectal cancer (CRC) patients with synchronous liver metastasis were given an intravenous injection of ^{99m}Tc tilmanocept followed by SPECT/CT imaging. ^{99m}Tc tilmanocept was administered at a dose of 50 μg/2mCi ^{99m}Tc to the first subject. A second subject received ^{99m}Tc tilmanocept administered at a dose of 200 μg/2mCi ^{99m}Tc. Additionally, each subject was initially screened using PET/CT imaging after FDG administration to identify hepatic lesions. The ^{99m}Tc tilmanocept was then administered and then PET/CT imaging of the subjects' abdomens were taken 4-6 hours after the injection. A safety follow-up was conducted between 2 to 8 days after the injection.

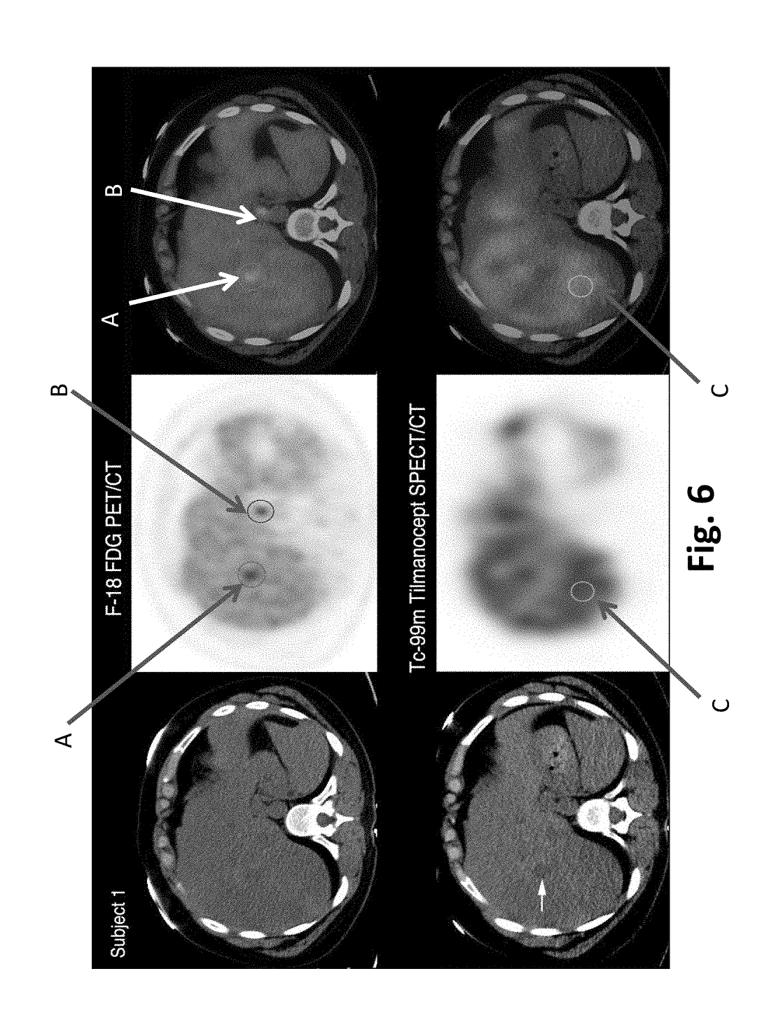

The first subject was a 26-year-old female with metastatic CRC with synchronized localization and disseminated liver metastases. Previous PET/CT (pre-treatment) scanning of the first subject showed 7-9 lesions. The first subject underwent round 9 of 12 chemotherapy sessions 10 days prior to FDG PET/CT and 14 days prior to receiving ^{99m}Tc tilmanocept. Two liver metastases were observed on an FDG PET/CT image of the subjects' abdomens, including the liver. There was localization of ^{99m}Tc tilmanocept in an area of larger metastasis than was observed with the FDG PET/CT. FIG. 6 shows the results achieved after administration of FDG PET/CT compared with the results achieved after administration of ^{99m}Tc tilmanocept. The results of the this example demonstrate the safety and tolerability of a dose of ^{99m}Tc tilmanocept administered intravenously.

IV injection of ^{99m}Tc tilmanocept was well-tolerated. No adverse drug reactions were observed. The FDG-PET/CT image (1A, red circles) shows a single metabolically active tumor focus in the right hepatic lobe. Incidental inflammatory activity is identified in the distal esophagus (1A, blue circle). The ^{99m}Tc tilmanocept-SPECT/CT image (1B, green circles), in addition to prior histological assessments in other tumors, suggests the presence of TAMs around the metastasis (CD206+ macrophages).

Circles labelled A in FIG. 6 show a single metabolically active tumor focus in the right hepatic lobe of the liver of subject one obtained from the administration and imaging of FDG. Circles labeled B shown in FIG. 6 correspond with distal esophagus incidental inflammatory activity obtained from the imaging of administration of FDG. The circles labelled C shown in FIG. 6 show the presence of TAMS around the liver metastasis, which is based on the prior histological assessment of other tumors.

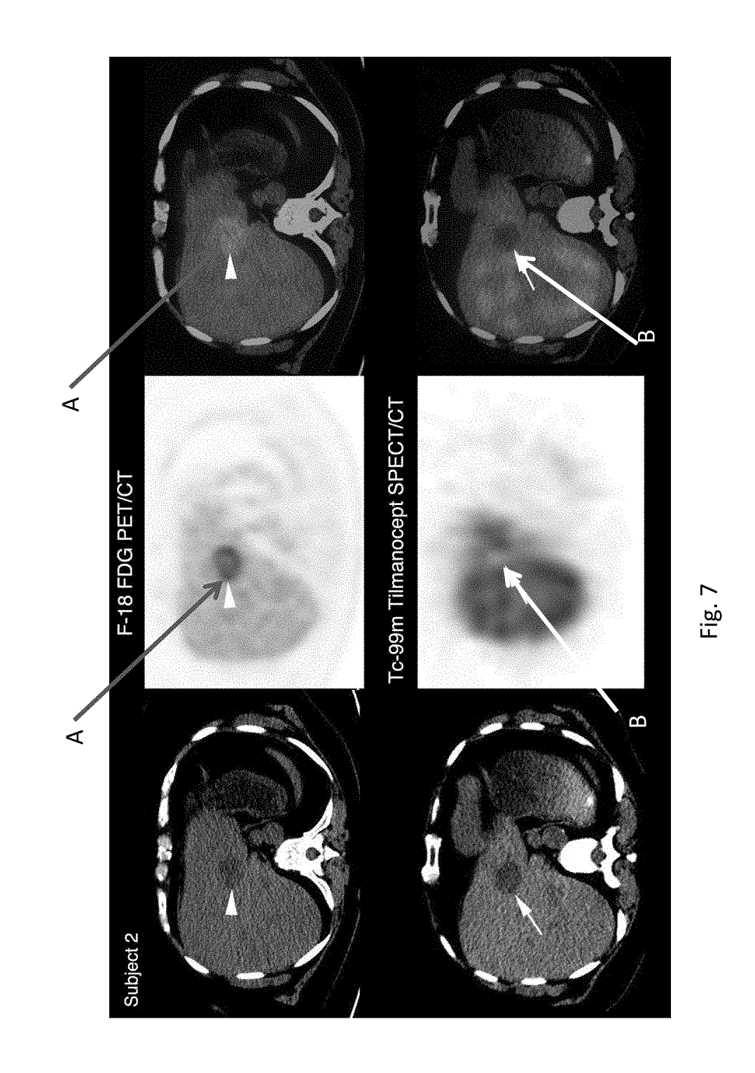

The second subject was a 59-year-old female with metastatic colorectal cancer with synchronized localization and disseminated liver metastases. FDG PET/CT images were obtained 10 days following the subject's second round of chemotherapy, where one liver metastasis was noted. The second subject underwent her third round of chemotherapy 10 days prior to receiving ^{99m}Tc tilmanocept. Localization around the metastasis was noted. FIG. 7 shows the comparative results obtained from administering FDG compared to administering ^{99m}Tc tilmanocept. The circles identified with arrows labeled as A in FIG. 7 show the identification of tumor cells obtained using F-18 FDG PET/CT imaging. The images shown in the lower half of FIG. 7 contain the images obtained from imaging using ^{99m}Tc tilmanocept. The arrows labeled B in FIG. 7 point to the foci of the tumor mass, and the surrounding darker area shows the presence of TAMS around metastases in the liver. In this experiment, the prior histological assessment allowed for the conclusion that these darker areas were, in fact, TAMS around metastases, demonstrating the improved ability of the instant invention to be able to identify metastases and tumor foci when imaging using ^{99m}Tc tilmanocept and other compositions described in the disclosure.

Mannosylated constructs such as the ones discussed herein, including ^{99m}Tc tilmanocept, and FDG are both molecular markers relevant to tumor biology and progressions. The results shown in FIG. 6 show that ^{99m}Tc tilmanocept binding may detect TAMS surrounding liver metastases. The diagnostic, prognostic, and therapeutic implications of TAM imaging in metastasized liver CRC remains to be determined, but mannosylated-based agents may serve to aid in targeting TAMs with a therapeutic payload, where response may be directly monitored through mannosylated-construct imaging.

While several compositions and methods for the detection and treatment of tumors have been discussed in detail above, it should be understood that the compositions, features, configurations, and methods of using the compositions discussed are not solely limited to the contexts provided above.

1-54. (canceled)

55. A composition used to detect one or more tumors wherein the composition comprises a mannosylated dextran compound.

56. The composition of claim 55, wherein the detection is achieved through localization of mannosylated dextran constructs to tumor associated macrophages (TAMs), myeloid derived suppressor cells (MDSC) and other tumor associated immune cells that express CD206.

57. A composition used to image a tumor, wherein the composition is a mannosylated composition without a dextran backbone.

58. The composition of claim 55, wherein the imaging is for a tumor that is not associated with breast cancer.

59. The composition of claim 55 comprising: ^{99m}Tc-tilmanocept, wherein said composition is used for imaging one or more tumors.

60. The composition of claim 55, wherein the at least one other sugar moiety can be added to the at least one leash, wherein the at least one other sugar is not occupied by either mannose moiety, diagnostic, or therapeutic moiety.

61. The composition of claim 55, wherein the composition further comprises a therapeutic agent.

62. A method of imaging a tumor comprising:
administering a compound to a subject comprising a dextran backbone having one or more CD206 targeting moieties and one or more diagnostic moieties attached thereto;
and imaging said subject using single-photon emission computed technology (SPECT) or positron-emission tomography (PET) without x-ray based computed technology (CT);
wherein an image comprises visual indications of uptake of said compound in one or tumors in a subject.

63. The method of claim 62, wherein the imaging of a subject can be done using planar gamma imaging wherein an image comprises visual indications of uptake of said compound in one or more tumors in the subject.

64. The method of claim 62, wherein the method is for imaging tumors that are not a result of breast cancer.

65. The method of claim 62, wherein the compound further comprises a therapeutic agent.

66. A method of imaging a tumor comprising:
administering a compound to a subject comprising a dextran backbone having one or more CD206 targeting moieties and one or more therapeutic moieties attached thereto;
and imaging said subject using single-photon emission computed technology (SPECT) or positron-emission tomography (PET) with x-ray based computed technology (CT) wherein an image comprises visual indications of uptake of said compound in one or tumors in a subject.

67. The method of claim 66, wherein the imaging of a subject can be done using planar gamma imaging wherein an image comprises visual indications of uptake of said compound in one or more tumors in the subject.

68. The method of claim 66, wherein the method is for imaging tumors that are not a result of breast cancer.

69. A method using the composition of claim 55, wherein the method does not include targeting dendritic cells.

70. A method comprising the steps of:
administering a composition comprising a carbohydrate backbone having one or more CD206 targeting moieties and one or more diagnostic moieties to a subject;
imaging the subject using SPECT/CT;
and determining the presence of tumor-associated macrophages.

71. The method of claim 70, further comprising the step of quantifying the number of tumor-associated macrophages.

72. The method of claim 70, further comprising the step of identifying the presence of tumors.

73. The method of claim 70, further comprising the step of identifying metastases of previously-existing tumors.

74. The method of claim 70, further comprising the step of administering a composition as described herein comprising a therapeutic agent for administering a therapeutic agent to a tumor.

75. The method of claim 70, further comprising the step of administering a composition as described herein comprising a therapeutic agent for administering a therapeutic agent to a metastasized tumor.

76. The method of claim 70, wherein the administration is intravenous.

77. The composition of claim 61, wherein the composition comprises a diagnostic moiety selected from the group consisting of ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr.

78. The composition of claim 61, wherein the diagnostic moiety is ⁶⁸Ga.

79. The composition of claim 61, wherein the diagnostic moiety is ^{99m}Tc.
